(19) United States
(12) Patent Application Publication
(10) Pub. No.: US 2018/0050090 A1
(43) Pub. Date: Feb. 22, 2018

(54) COMPOSITIONS AND METHODS FOR SENSITIZING CELLS TO TRAIL-INDUCED APOPTOSIS

(71) Applicant: THE JOHNS HOPKINS UNIVERSITY, Baltimore, MD (US)

(72) Inventors: Seulki Lee, Elkridge, MD (US); Martin G. Pomper, Baltimore, MD (US); Magdalena Swierczewska, Columbia, MD (US); Yumin Oh, Elkridge, MD (US)

(21) Appl. No.: 15/559,108

(22) PCT Filed: Mar. 15, 2016
(86) PCT No.: PCT/US2016/022462

§ 371 Date: Sep. 18, 2017

Related U.S. Application Data
(60) Provisional application No. 61/134,674, filed on Jul. 11, 2008.

(51) Int. Cl. A61K 38/19; A61K 47/60

(52) U.S. Cl. CPC A61K 38/191; A61K 33/24; A61K 2300/00; A61K 47/60

(57) ABSTRACT

The present invention comprises pegylated TRAIL peptides and their use in conjunction with various TRAIL sensitizing agents in tumor homing nanoparticle formulations for use in the treatment of cancer in a subject.

### REFERENCE TO RELATED APPLICATIONS

This application claims the benefit of U.S. Provisional Patent Application No. 62/134,674, filed on Mar. 18, 2015, which is hereby incorporated by reference for all purposes as if fully set forth herein.

### STATEMENT OF GOVERNMENTAL INTEREST

This invention was made with government support under grant no. EB013450, CA130460, PC131920 awarded by the National Institutes of Health. The government has certain rights in the invention.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Mar. 15, 2016, is named P13440-02_ST25.txt and is 1,428 bytes in size.

### BACKGROUND OF THE INVENTION

Recombinant human tumor necrosis factor (TNF)-related apoptosis inducing ligand (rhTRAIL) and its agonistic antibodies have been under intense focus as crucial, molecularly targeted, antitumor biologics. Unlike conventional anticancer agents and even other TNF family members, rhTRAIL selectively transduces apoptotic signals by binding to death receptors (DRs) that are widely expressed in most cancers, TRAIL-R1/DR4 and TRAIL-R2/DR5, while sparing normal cells. This high tumor specificity along with broad applicability across multiple cancer types and proven safety in humans make TRAIL an ideal candidate for cancer therapy. However, recent clinical trials of rhTRAIL, e.g. dulanermin, or humanized DR agonistic monoclonal antibodies, tested as either a monotherapy or combined with anticancer agents have failed to demonstrate benefits in cancer patients compared with historical controls. The disappointing results raise concerns for the therapeutic implications of rhTRAIL.

The primary challenge in TRAIL-based therapy is natural resistance. The majority of primary cancer cells are TRAIL-resistant. Mechanisms of TRAIL resistance are distinct among cancer cell types; however, they commonly comprise of: reduced cell surface DR expression, inhibited caspase-8 activation—the initiator caspase, up-regulated anti-apoptotic molecules such as Bcl-2 and the inhibitors of apoptosis (IAP) family proteins, and reduced expression of pro-apoptotic markers like Bax/Bak. The role of diverse molecules like anticancer agents and natural compounds in sensitizing TRAIL-resistant cancer cells has been investigated and introduced as an addition to TRAIL monotherapy. TRAIL-based combinations were well validated in vitro and in a few in vivo cancer models; however, they fail to demonstrate a similar synergy in cancer patients. The critical reasons for ineffectiveness of rhTRAIL combination in humans are not clearly explained in the literature. This implies a need for alternative approaches to realize rhTRAIL combination therapy in the clinic.

In addition to TRAIL-resistance, rhTRAIL has an extremely short half-life in physiological conditions, 3-5 min in rodents and less than 30 minutes in humans. It is widely accepted that wild-type proteins with short half-lives do not exhibit similar biological potency in physiological conditions as those tested in vitro. Use of a more stable form of rhTRAIL with an extended half-life would be expected to improve TRAIL action in physiological conditions, particularly for a biologic with an exceptionally short half-life like TRAIL.

### SUMMARY OF THE INVENTION

The present inventors have developed a series of long-acting PEGylated TRAILs (TRAIL_{PEG}) by PEGylating an isoleucine-zipper-tagged TRAIL (iLZ-TRAIL), a TRAIL variant that is known to more potent than non-tagged rhTRAIL. PEGylation is considered the gold standard for half-life extension and a highly efficient commercial strategy as proven by PEGylated interferons and other FDA-approved biologics. TRAIL_{PEG} has increased stability over rhTRAIL with a significantly longer circulation half-life in rats. As a result, TRAIL_{PEG} demonstrated superior in vivo anticancer potencies in xenografts bearing TRAIL-sensitive HCT116 colon cancer tumors over iLZ-TRAIL. However, increasing circulation time of TRAIL cannot be a solution for targeting primary tumors associated with TRAIL resistance at the molecular level.

The present inventors believed that TRAIL can have clinical efficacy in cancer by simultaneously addressing two key limitations, TRAIL resistance and its short half-life. First, a TRAIL sensitizer was selected in TRAIL-resistant colon cancer cells through cell-based screening and TRAIL and apoptotic signals were explored at the molecular level. Next, the selected TRAIL sensitizer alone or formulated with tumor-homing polymer nanoparticles were systemically administered to xenografts bearing TRAIL-resistant tumors followed by TRAIL_{PEG} administration to investigate a synergistic effect on TRAIL-induced apoptosis in vivo. Lastly, the inventors show the necessary conditions to potentiate anticancer efficacy of TRAIL with a select, tumor-homing TRAIL sensitizer and TRAIL variant in vivo. These studies demonstrate that strategies that address the short half-life of TRAIL in vivo alone or TRAIL resistance alone are not effective and hence may explain the disappointing clinical results of TRAIL-based cancer therapies thus far.

In accordance with an embodiment, the present invention provides a method for treating cancer in a subject comprising: 1) identification of the tumor's TRAIL sensitivity; administering to the subject a nanoparticle comprising an effective amount of one or more TRAIL sensitizing compounds; and administering to the subject an effective amount of a pegylated TRAIL peptide to induce apotosis in the cancer of the subject.

In accordance with an embodiment, the present invention provides a composition comprising pegylated TRAIL peptide.

In accordance with an embodiment, the present invention provides composition comprising a nanoparticle comprising an effective amount of one or more TRAIL sensitizing compounds.

### BRIEF DESCRIPTION OF THE DRAWINGS

FIGS. 1A-1E: PEGylation extends the biological half-life of TRAIL, but does not enhance apoptosis in TRAIL-resistant cancer cell lines. (A) Serum concentration versus time profiles following single intravenous dosing (dose=12.5 μg/kg, protein-based) with iLZ-TRAIL and TRAILPEG in cynomolgus monkeys (n=2). (B) HCT116 xenografts were established and mice were intravenously treated when the tumor was palpable with four rounds of saline, iLZ-TRAIL (200 μg) or TRAILPEG (200 μg, protein-based). Tumor volumes were determined by caliper measurements (n=5/group). (C) TUNEL staining of harvested tumors. At the end of the study, animals were sacrificed and tumors were processed and analyzed for TUNEL staining. Fluorescence images were acquired under a confocal microscope and overlaid with Hoechst 33258 staining. (D) Human tumor cell lines: colon (HT-29, SW620, HCT116), prostate (PC3), breast (MDA-MB-231, MCF7) and lung (A549) and normal human cell line: kidney (HEK293T) were collected and examined for their sensitivities to iLZ-TRAIL and TRAILPEG by cell death assay. Cells were treated with TRAIL variants (1 μg/mL, protein-based) for 24 h and cell death rates were measured by MTT assay (n=3). (E) Western blot showing the processing of caspase-8 (Casp-8) and the cleaved PARP-1 (Cl. PARP-1), the caspase-3 substrate, in select cells treated with iLZ-TRAIL or TRAILPEG. β-actin was used as a protein loading control. *P<0.001 vs. control group (without any treatment). Values indicate means±S.E.

FIGS. 2A-2F: Doxorubicin (DOX) initiates a caspase cascade and induces apoptosis when combined with TRAILPEG in TRAIL-resistant cancer cell lines. (A) DNA damaging agents sensitize TRAIL-induced apoptosis in HT-29 cells. HT-29 cells were treated with sublethal doses of doxorubicin (DOX, 2 μg/mL), 5-fluorouracil (5-FU, 10 μg/mL), cisplatin (CIS, 2 μg/mL) and irinotecan (IRINO, 2.9 μg/mL) for 24 h and further incubated with TRAILPEG (1 μg/mL) for an additional 24 h. The cell death rates were measured by MTT assay (n=3). *P<0.001 vs. cells treated with cytotoxic agent only (Ctrl). Values indicate means±S.D. (B) The cell extracts were prepared and the levels of proteins were examined by western blotting; cleaved PARP-1 (Cl. PARP-1), caspase-8 (Casp-8), cleaved Casp-8, c-Jun and phospho-p53 (p-p53). β-actin was used as a protein loading control. (C) A combination of TRAILPEG and DOX but not drug alone sensitizes TRAIL-induced apoptosis in various TRAIL-resistant cells, HT-29 (colon), MDA-MB-231 (breast), A549 (lung), and PC3 (prostate), as in TRAIL-sensitive HCT116 (colon) cancer cells. (D) DISC formation in HT-29 cells. HT-29 cells were left untreated or stimulated with 500 ng/ml of TRAILFlag for 1 h. The lysates were immunoprecipitated with FLAG (M2) and analyzed by Western blotting using DR4 and DR5 antibodies. WCL: Whole cell lysates. (E and F) DR5 induction in HT-29 cells by DOX. (E) HT-29 cells were transfected with DR5 siRNA for 48 h and the cells were left untreated or incubated with DOX for an additional 24 h. Cell extracts were examined by western blotting for DR5 using anti-DR5 and anti-β-actin antibodies. (3-actin was used as a protein loading control. (F) HT-29 cells were treated with DOX for 24 h and the cell extracts were examined for mRNA levels of DR4 and DR5 using gene-specific primers by qRT-PCR analysis.

FIGS. 3A-3F: When combined with TRAILPEG, DOX synergizes TRAIL-induced apoptosis in HT-29 cells through DR5 upregulation and partially by JNK-mediated apoptosis. (A) Western blotting analysis of HT-29 cells treated with TRAILPEG (1 μg/mL) and DOX (2 μg/mL) alone or in combination with different incubation times. The cell extracts were prepared and the levels of DR4, DR5 and cleaved caspase-8 (Cl. Casp-8) and caspase-3 (Cl. Casp-3) were examined. (B) The relative fold increase of cleaved caspase-3, caspase-8, DR4 and DR5 expressions from control group (no TRAILPEG (1 μg/mL) and DOX (2 μg/mL) treatment). (C) The effect of upregulated DR5 on TRAIL-induced cell death in HT-29 cells. Cells were treated with DOX (2 μg/mL) and TRAILPEG (1 μg/mL) alone or in combination with or without DR5-A (2 μg/mL, DR5 antagonist peptide) pretreatment. Cell death rates were measured by MTT assay (n=3). *P<0.001 vs. DR5 neutralized group. (D) Western blotting analysis of cells as treated in (C). Cleaved caspases, PARP-1, DR4 and DR5, BCL2, BCL-XL and β-actin (loading control) was measured. (E) The effect of JNK on TRAIL-induced cell death in HT-29 cells. Cells were treated with DOX (2 μg/mL) and TRAILPEG (1 μg/mL) alone or in combination with or without SP600125 (JNK inhibitor, 20 μM) pre-treatment. Cell death rates were measured by MTT assay (n=3). *P<0.001 vs. JNK activity inhibited group. (F) Cleaved caspases, PARP-1, phosphorylated JNK and DR5 western blot analysis of cells in (E). β-actin was measured as a loading control.

FIGS. 4A-4E: HAC/DOX but not free DOX accumulates in tumors for a sustained period of time and potentiates caspase cascade when combined with TRAILPEG. (A) Upper; schematic diagram of HAC/DOX, hyaluronic acid-based conjugate (HAC) carrying doxorubicin in the core and FITC dye molecules labeled on the surface for fluorescence microscopy. Lower; a chemical structure of HAC, hyaluronic acid chemically conjugated with cholanic acid. (B) Cancer cells were examined for their CD44 expression. The cell extracts were prepared and the levels of CD44 were examined by western blotting. (C) HAC/DOX rapidly internalizes and releases DOX in HT-29 cells. HT-29 cells were incubated with HAC/DOX (2 μg/mL, doxorubicin-based) for 10 min and 60 min. Fluorescence images were acquired under a confocal microscope and overlaid with Hoechst 33258 staining. HAC; green, DOX; red, and nucleus; blue. FACS analysis described in FIG. 9. (D) DOX concentration in the harvested tumors following single intravenous dosing of DOX (7 mg/kg) and HAC/DOX (7 mg/kg, DOX-based) in HT-29 xenografts. When tumors reached a diameter of 300 mm3, mice were intravenously treated with DOX and HAC/DOX. At the indicated time points, mice were sacrificed and the tumor concentration of doxorubicin were measured by RP-HPLC method followed by extraction recovery (n=3). Values indicate means±S.D. (E) The uptake and distribution of doxorubicin in tumor tissues. Representative fluorescence images of tumor sections demonstrate the high accumulation of doxorubicin after HAC/DOX injection. Nucleus; blue (DAPI), doxorubicin; red (TRITC).

FIGS. 5A-5E: Simultaneous treatment of TRAILPEG and HAC/DOX initiates apoptosis and reduces tumor growth in TRAIL-resistant tumors in vivo. (A) Lysates of HT-29 tumors from mice treated with TRAILPEG (200 μg per mouse) and HAC/DOX (7 mg/kg, DOX-based) alone or in combination were western blotted for death receptors (DR5, DR4), cleaved caspases and β-actin (loading control) expression analysis. (B, C) The relative fold increase of DR and caspase expressions. (D) Mice bearing approximately 150 mm3 HT-29 tumors were intravenously treated with vehicle, TRAILPEG (200 μg) alone, TRAILPEG (200 μg) combined with DOX (7 mg/kg) or HAC/DOX (7 mg/kg, DOX-based) every 3 days starting at day 15 for a total of 3 doses. Tumor volumes were determined by caliper measurements (n=5/group). Values indicate means±S.E.M. (E) Survival rate curve of mice treated in (D).

FIGS. 6A-6C: (A) Human tumor cell lines: colon (HT-29, SW620, HCT116), prostate (PC-3), breast (MDA-MB-231R, MCF7) and lung (A549) and normal human cell line: kidney (HEK293T) were collected and examined for their sensitivities to iLZ-TRAIL and TRAILPEG by cell death assay. Cells were treated with TRAIL variants (1 μg/mL, protein-based) for 3 h and cell death rates were measured by MTT assay (n=3). (B) HT-29 cells were treated with low doses of doxorubicin (DOX, 0.5 μg/mL), 5-fluorouracil (5-FU, 1 μg/mL), cisplatin (CIS, 0.5 μg/mL) and irinotecan (IRINO, 0.59 μg/mL) for 24 h and further incubated with TRAILPEG (1 μg/mL) for an additional 24 h. The cell death rates were measured by MTT assay (n=3). Controls were cells treated with cytotoxic agent only. (C) A combination of TRAILPEG and DOX sensitizes TRAIL-induced apoptosis in various TRAIL-resistant cells. Cells were treated with doxorubicin (DOX, 2 μg/mL) for 24 h and further incubated with TRAILPEG (1 μg/mL, protein-based) for 3 h and cell death rates were measured by MTT assay (n=3).

FIGS. 7A-7B: (A) Chemical structures, RP-HPLC and MALDI-TOF mass spectra of DR5 specific binding peptide, DR5-A and (B) FITC-labeled DR5-A, FITC-DR5-A.

FIGS. 8A-8C: (A) Characterization of FITC-DR5-A. HT-29 cells were treated with FITC-DR5-A for 10 min or pretreated with anti-DR5 antibody for 60 min followed by FITC-DR5-A treatment and captured under a confocal microscope. (B) HCT116 cells were treated with DR5-A followed by TRAILPEG for 3 h. The cell lysates were examined for cellular apoptosis by western blotting for indicated antibodies. Cl.: cleaved. (C) HCT116 cells were treated with DR5 antagonistic peptide followed by TRAILPEG or DR5 agonistic antibody for 3 h. The cell lysates were examined by Western blotting for the apoptosis marker, cleaved PARP-1 (Cl. PARP-1), the caspase-3 substrate.

FIGS. 9A-9C. (A) Diameter measurements of HAC and HAC/DOX, DOX loaded HAC, as measured by a Malvern Zetasizer Nano Z. (B) FACS analysis to determine the levels of HAC internalization after HT-29 cells were treated with HAC/DOX or HAC/FITC for 1 h. Samples were analyzed using a flow cytometer. (C) Intracellular staining of HT-29 cells after being treated with HAC/DOX or HAC/FITC at indicated times and captured under a confocal microscope.

FIGS. 10A-10C: (A) Quantification of the accumulated doxorubicin after HAC/DOX injection in FIG. 4E. (B) When HT-29 xenografts tumors reached a diameter of 200 mm3, mice were intravenously treated with DOX (low: 2 mg/kg, or high: 7 mg/kg) and HAC/DOX (2 or 7 mg/kg, DOX-based) followed by TRAILPEG. The tissue extracts were prepared and the activation of caspases (Casp-8, -9, and -3) were examined by western blotting. (B) Quantification of the cleaved caspases in (B).

### DETAILED DESCRIPTION OF THE INVENTION

In accordance with an embodiment, the present invention provides a method for treating cancer in a subject comprising utilizing rhTRAIL with an extended half-life and effectively sensitizing TRAIL-resistant tumors through tumor-homing TRAIL sensitizers.

In accordance with another embodiment, the present invention provides a method of treating cancer in a subject comprising administering to the subject a TRAIL peptide that is pegylatated and a composition comprising a TRAIL sensitizing agent and a nanoparticle.

In accordance with an embodiment, the present invention provides a method for treating cancer in a subject comprising: 1) identification of the tumor's TRAIL sensitivity; administering to the subject a nanoparticle comprising an effective amount of one or more TRAIL sensitizing compounds; and administering to the subject an effective amount of a pegylated TRAIL peptide to induce apotosis in the cancer of the subject.

In accordance with an embodiment, the present invention provides a composition comprising pegylated TRAIL peptide.

In accordance with an embodiment, the present invention provides composition comprising a nanoparticle comprising an effective amount of one or more TRAIL sensitizing compounds.

Various forms of TRAIL receptor agonists (TRAs) or death receptor agonist (DRAs) have been utilized—antibodies, peptides, recombinant proteins for universal cancer therapy. They are very useful as therapeutics for cancer because of their selectivity for diseased cells, but there are many shortcomings that need to be avoided. Most prominently the following three features need to be overcome for effective cancer therapy by TRAs: cancer cell resistance, short half-life and trimerization efficacy of TRAs. Methods that overcome one or two of these disadvantages have been published and are mostly limited to in vitro demonstration. The delivery strategy introduced in the present invention, addresses all three in physiological conditions.

In accordance with one or more embodiments, the treatment strategy can apply a diverse set of carriers, TRAIL sensitizers and TRAs (recombinant human TRAIL variants, antibodies, DR peptide, etc.). In an embodiment, as an example, a biomaterial nanoparticle, a common chemotherapeutic, and a PEGylated TRAIL protein is utilized as the carrier, sensitizer and TRA, respectively.

In some embodiments, a TRA: long-acting no more than one dose per day, induces effective trimerization, and is stable in vivo.

In another embodiment, in humans, TRAIL binds two proapoptotic death receptors (DRs), TRAIL-R1 and -R2 (TNFRSF10A and 10B), as well as two other membrane receptors that do not induce death and instead may act as decoys for death signaling. TRAIL binding to its cognate DRs induces formation of a death-inducing signaling complex, ultimately leading to caspase activation and initiation of apoptosis.

In some embodiments, TRAs can be: TRAIL peptide; or an agonistic TRAIL receptor binding fragment or variant thereof. A nucleic acid and amino acid sequence for human TRAIL are known in the art (UniProtKB database accession no. P50591).

In some embodiments, preferably, the TRAIL is a soluble TRAIL

As used herein, soluble TRAIL means, for example, a fragment of full-length TRAIL without the cytoplasmic domain and the transmembrane domain or a functional fragment. In some embodiments the TRAIL of the present invention can have 50, 60, 70, 75, 80, 85, 90, 95, 96, 97, 98, 99, or more than 99% sequence identity human TRAIL

In some alternative embodiments, TRAIL can encompass a functional fragment that can agonize signaling through TRAIL-R1 and/or TRAIL-R2.

It will be understood by those of skill in the art that a consensus extracellular domain for human TRAIL comprises amino acids 39-281.

In some other embodiments, the TRAIL peptide can include amino acids 39-281, 41-281, 91-281, 92-281, 95-281, and 114-281, or a functional fragments or variants thereof.

As used herein, the functional fragments of TRAIL, include amino acids 132-281, amino acids 95-281, or amino acids 114-281 to include C-terminal of TRAIL that includes receptor binding domain.

In accordance with an embodiment, variants can have one or more substitutions, deletions, or additions, or any combination.

In some embodiments, TRAIL ligands or agonists can form, a multimer, preferably a trimer. The trimer can be a homotrimer, or a heterotrimer, for example.

In some embodiments, a TRAIL analogue, or an agonistic TRAIL receptor can include a binding fragment or variant.

In some embodiments, TRAIL can be a recombinant or native TRAIL

The embodiments of TRAIL can include increased affinity or specificity for one or more agonistic TRAIL receptors (e.g., TRAIL-R1 (DR4) and/or TRAIL-R2 (DR5)), reduced affinity or specificity for one or more antagonistic or decoy TRAIL receptors (e.g., receptors DcR1 and DcR2) or a combination compared to wildtype or endogenous TRAIL

In some embodiments, TRAIL peptides or proteins can include TRAIL fusion proteins. The TRAIL fusion proteins may contain a domain that functions to dimerize or multimerize two or more fusion proteins.

In some embodiments, the TRAIL peptides or proteins form dimers or multimers that are formed can be homodimeric/homomultimeric or heterodimeric/heteromultimeric.

The TRAIL peptides of the present invention can also comprise one or more linker domains that can either be a separate domain, or alternatively can be contained within one of the other domains (TRAIL polypeptide or second polypeptide) of a fusion protein.

In some embodiments, TRAIL-mimic compositions can include three TRAIL-protomer subsequences combined in one polypeptide chain, termed the single-chain TRAIL-receptor-binding domain (scTRAIL-RBD).

In some embodiments, TRAIL fusion proteins have a multimerization domain, such as a dimerization or trimerization domain, or a combination thereof that can lead to, for example, dimeric, trimeric, or hexameric molecule.

In other embodiments, the fusion protein that facilitates trimer formation includes a receptor binding fragment of TRAIL amino-terminally fused to a trimerizing leucine or isoleucine zipper domain.

In alternative embodiments, the present methods can use an antibody that specifically binds to a TRAIL receptor, including, for example, recombinant antibodies, fragments of antibodies, single-chain antibodies, monovalent antibodies, single-chain antibody variable fragments, divalent single-chain variable fragments, diabodies, triabodies, tetrabodies, human or humanized antibodies, hybrid antibodies/chimeric antibodies, TRA conjugates or complexes, conjugate molecules linked to the TRA, including polymers or copolymers, and polyalkylene oxides (e.g. PEG).

In some embodiments, derivatives of PEG include, but are not limited to, methoxypolyethylene glycol succinimidyl propionate, methoxypolyethylene glycol N-hydroxysuccinimide, methoxypolyethylene glycol aldehyde, methoxypolyethylene glycol maleimide and multiple-branched polyethylene glycol.

In accordance with the inventive methods, the PEG molecular weight in the invention can be within about, 1-100 kDa, can be linear or branched, can comprise biopolymers, polypeptides, hyaluronic acid, chitosan, albumin, chondroitin sulfate, and XTEN technology (Versartis).

In accordance with one or more embodiments, the TRAIL_{PEG} complex can be complexed with a carrier (e.g. to form nanoparticle). Other half-life extension technologies/controlled release technologies known in the formulation arts can be used with the present invention.

The compositions of the present invention can be combined with targeting moieties, such as, for example, antibodies, small molecules, peptides, conjugates to improve purification, Tag-removal, facilitate small molecule attachment, solubility or a combination thereof, elastin-like polypeptides and the Sortase A (SrtA) transpeptidase, SUMO tags, His tags, FLAG tags and MYC tags.

In some embodiments the expression or solubility enhancing amino acid sequence can be manipulated using one or more of the following: maltose-binding protein (MBP), glutathione S-transferase (GST), thioredoxin (TRX), NUS A, ubiquitin (Ub), and SUMO

In other embodiments, linkers or spacers (e.g. peptides) to link domains, regions or sequences to each other.

In accordance with an embodiment, the present invention provides a specific TRA sensitizer: upregulates death receptors (commercially available or novel). These sensitizers can increase targeting to or accumulation of TRA to site of interest, and are preferably chemotherapeutic agents on their own.

It has been shown that although TRAIL is capable of inducing apoptosis in tumor cells of diverse origin, a majority of tumor cells are resistant to the apoptotic effects of TRAIL, suggesting that TRAIL alone may be ineffective for the treatment of these cancers. Furthermore, several studies have shown that chemotherapeutic drugs {e.g. cisplatin, carboplatin, etoposide, camptothecin, paclitaxel, vincristine, and vinblastine, doxorubicin, gemcitabine and 5-fluorouracil) can sensitize TRAIL-resistant breast, prostate, colon, bladder, and pancreatic cancer cells to TRAIL in vitro and in vivo, indicating that combination therapy may be a possibility. Furthermore, it was shown that chemotherapeutic drugs not only induce death receptors in vitro, but also in tumor xenografts in nude mice, suggesting that these conventional chemotherapeutic drugs might enhance the cytotoxicity of TRAIL in humans. Several breast and prostate cancer cells are resistant to apoptosis by TRAIL, and chemotherapeutic drugs sensitize TRAIL-resistant cells to undergo apoptosis by up-regulating DR4 and/or DR5 and activating caspase. The chemotherapeutic drugs synergize with TRAIL in reducing tumor growth, inducing tumor-cell apoptosis and enhancing survival of tumor-bearing mice. Furthermore, it has been shown that chemotherapeutic drugs such as cisplatin, carboplatin, etoposide, camptothecin, doxorubicin, gemcitabine, 5-fluorouracil, paclitaxel, vincristine, and vinblastine can be used with TRAIL to kill TRAIL-sensitive and -resistant breast cancer cells. Sensitizing agents can include, for example, chemopreventative drugs, curcumin, and phytochemicals, naturally occurring antioxidant compounds (e.g. resveratrol)

In other embodiments, the methods of the present invention can include, but are not limited to, immunotherapies, gene therapies, anti-angiogenic agents, and chemotherapeutic agents, such as, for example, adriamycin, doxorubicin, 5-fluorouracil, cytosine arabinoside, cyclophosphamide, thiotepa, docetaxel, busulfan, cytoxin, taxol, paclitaxel, methotrexate, gemcitabine, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins, melphalan and other related nitrogen mustards.

In other embodiments, the methods of the present invention can include radiation therapies, biologics for cancer therapy, including, HERCEPTIN™ (trastuzumab), which may be used to treat breast cancer and other forms of cancer; RITUXAN™ (rituximab), ZEVALIN™ (ibritumomab tiuxetan), and LYMPHOCIDE™ (epratuzumab), which may be used to treat non-Hodgkin's lymphoma and other forms of cancer; GLEEVEC™ (imatinib mesylate), which may be used to treat chronic myeloid leukemia and gastrointestinal stromal tumors; and BEXXAR™ (tositumomab), which may be used for treatment of non-Hodgkin's lymphoma. Certain exemplary antibodies also include ERBITUX™; VECTIBIX™, IMC-C225; IRESSA™ (gefitinib); TARCEVA™ (ertinolib); KDR (kinase domain receptor) inhibitors; anti VEGF antibodies and antagonists (e.g., AVASTIN™ and VEGF-traps); anti-VEGF (vascular endothelial growth factor) receptor antibodies, peptibodies, and antigen binding regions; anti-Ang-1 and Ang-2 antibodies, peptibodies (e.g., AMG 386, Amgen Inc), and antigen binding regions; antibodies to Tie-2 and other Ang-1 and Ang-2 receptors; Tie-2 ligands; antibodies against Tie-2 kinase inhibitors; and CAMPATH™, (alemtuzumab).

Other therapies that can be combined with the inventive compositions and methods include use of HDAC inhibitors; anti-inflammatory agents; inhibitors of COX-2 and/or iNOS.

In accordance with an embodiment, the sensitizers can be administered prior to and/or subsequent to (collectively, “sequential treatment”), and/or simultaneously with (“concurrent treatment”) a specific binding agent of the present invention. Sequential treatment (such as pretreatment, post-treatment, or overlapping treatment) of the combination, also includes regimens in which the drugs are alternated, or wherein one component is administered long-term and the other(s) are administered intermittently. Components of the combination may be administered in the same or in separate compositions, and by the same or different routes of administration.

In some embodiments, tumor-homing carrier of a TRA sensitizer: targets tumors by specific ligands or enhanced permeability effect and delivers active sensitizer.

Carriers may covalently or non-covalently bind the TRA sensitizer. TRA sensitizer may be opro-drug. Preferably carries would be biodegradable. Products currently in development for tumor-homing or tumor targeted approaches include: microspheres; virosomes; engineered nanoparticles (e.g. Accurins™ by Bind Therapeutics); dendrimers; nanocrystals; block copolymer micelles; polymeric nanoparticles; albumin-bound (e.g. Abraxane®); PLGA nanoparticles; chitosan analog nanoparticles; PEG nanoparticles; targeting moieties can be peptides, antibodies, proteins and others compounds listed above.

In accordance with one or more embodiments, the compositions and methods can be used to trean various cancer indications. The present invention may be used to treat individual that has cancer, such as brain, lung, liver, spleen, kidney, lymph node, small intestine, pancreas, blood cell, bone, colon, stomach, breast, endometrium, prostate, testicle, ovary, central nervous system, skin, head and neck, esophagus, or bone marrow cancer. In some embodiments, the cancer is mesothelioma. In other embodiments said cancer is leukemia. In still other embodiments, the cancer is epithelial cancer. In still further embodiments, the bone marrow cancer is multiple myeloma. In still further embodiments, the individual has been identified as having a high risk for the development of cancer (see, for example, WO2008/094319 A2).

The cancers which can be treated by the methods of the invention include, but are not limited to, liver cancer, brain cancer, renal cancer, breast cancer, pancreatic cancer (adenocarcinoma), colorectal cancer, lung cancer (small cell lung cancer and non-small-cell lung cancer), spleen cancer, cancer of the thymus or blood cells (i.e., leukemia), prostate cancer, testicular cancer, ovarian cancer, uterine cancer, gastric carcinoma, head and neck squamous cell carcinoma, melanoma, and lymphoma. In some embodiments the cancer is non-small cell lung cancer (NSCLC) (see. WO2013/148877 A1).

“Treating” or “treatment” is an art-recognized term which includes curing as well as ameliorating at least one symptom of any condition or disease. Treating includes reducing the likelihood of a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder or condition, e.g., causing any level of regression of the disease; inhibiting the disease, disorder or condition, e.g., impeding its progress; and relieving the disease, disorder or condition, even if the underlying pathophysiology is not affected or other symptoms remain at the same level.

“Prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term, “carrier,” refers to a diluent, adjuvant, excipient or vehicle with which the therapeutic is administered. Such physiological carriers can be sterile liquids, such as water and oils, including those of petroleum, animal, vegetable or synthetic origin, such as peanut oil, soybean oil, mineral oil, sesame oil and the like. Water is a suitable carrier when the pharmaceutical composition is administered intravenously. Saline solutions and aqueous dextrose and glycerol solutions also can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents.

Polymer is used to refer to molecules composed of repeating monomer units, including homopolymers, block copolymers, heteropolymers, random copolymers, graft copolymers and so on. “Polymers” also include linear polymers as well as branched polymers, with branched polymers including highly branched, dendritic, and star polymers.

A monomer is the basic repeating unit in a polymer. A monomer may itself be a monomer or may be dimer or oligomer of at least two different monomers, and each dimer or oligomer is repeated in a polymer.

“Incorporated,” “encapsulated,” and “entrapped” are art-recognized when used in reference to a therapeutic agent, dye, or other material and a polymeric composition, such as a composition of the present invention. In certain embodiments, these terms include incorporating, formulating or otherwise including such agent into a composition that allows for sustained release of such agent in the desired application. The terms may contemplate any manner by which a therapeutic agent or other material is incorporated into a polymer matrix, including, for example, attached to a monomer of such polymer (by covalent or other binding interaction) and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to the incorporation of a therapeutic agent or other material and at least one other therapeutic agent or other material in a subject composition.

More specifically, the physical form in which any therapeutic agent or other material is encapsulated in polymers may vary with the particular embodiment. For example, a therapeutic agent or other material may be first encapsulated in a microsphere and then combined with the polymer in such a way that at least a portion of the microsphere structure is maintained. Alternatively, a therapeutic agent or other material may be sufficiently immiscible in the polymer of the invention that it is dispersed as small droplets, rather than being dissolved in the polymer. Any form of encapsulation or incorporation is contemplated by the present invention, in so much as the sustained release of any encapsulated therapeutic agent or other material determines whether the form of encapsulation is sufficiently acceptable for any particular use.

Pharmaceutically acceptable salts are art-recognized, and include relatively non-toxic, inorganic and organic acid addition salts of compositions of the present invention, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di-, or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenthylamine; (trihydroxymethyl) aminoethane; and the like, see, for example, J. Pharm. Sci., 66: 1-19 (1977).

It will be understood that “substitution” or “substituted with” includes the implicit proviso that such substitution is in accordance with the permitted valency of the substituted atom and the substituent, and that the substitution results in a stable compound, e.g., which does not spontaneously undergo transformation, such as by rearrangement, cyclization, elimination, or other reaction.

Methods for the synthesis of the polymers described above are known to those skilled in the art, see, e.g., Concise Encyclopedia of Polymer Science and Polymeric Amines and Ammonium Salts, E. Goethals, editor (Pergamen Press, Elmsford, N.Y. 1980). Many polymers, such as poly(acrylic acid), are commercially available. Naturally occurring polymers can be isolated from biological sources as known in the art or are commercially available. Naturally occurring and synthetic polymers may be modified using chemical reactions available in the art and described, for example, in March, “Advanced Organic Chemistry,” 4th Edition, 1992, Wiley-Interscience Publication, New York.

A composition of this invention may further contain one or more adjuvant substances or the like. Such additional materials may affect the characteristics of the resulting composition. For example, fillers, such as bovine serum albumin (BSA) or mouse serum albumin (MSA), may be associated with the polymer composition. In certain embodiments, the amount of filler may range from about 0.1 to about 50% or more by weight of the composition. Incorporation of such fillers may affect the sustained release rate of any encapsulated substance. Other fillers known to those of skill in the art, such as carbohydrates, sugars, starches, saccharides, celluloses and polysaccharides, including and sucrose, may be used in certain embodiments in the present invention.

Buffers, acids and bases may be incorporated in the compositions to adjust pH. Agents to increase the diffusion distance of agents released from the composition may also be included.

The charge, lipophilicity or hydrophilicity of a composition may be modified by employing an additive. For example, surfactants may be used to enhance miscibility of poorly miscible liquids. Examples of suitable surfactants include dextran, polysorbates and sodium lauryl sulfate. In general, surfactants are used in low concentrations, generally less than about 5%.

The specific method used to formulate the novel formulations described herein is not critical to the present invention and can be selected from a physiological buffer (Feigner et al., U.S. Pat. No. 5,589,466 (1996)).

Therapeutic formulations of the product may be prepared for storage as lyophilized formulations or aqueous solutions by mixing the product having the desired degree of purity with optional pharmaceutically acceptable carriers, diluents, excipients or stabilizers typically employed in the art, i.e., buffering agents, stabilizing agents, preservatives, isotonifiers, non-ionic detergents, antioxidants and other miscellaneous additives, see Remington's Pharmaceutical Sciences, 16th ed., Osol, ed. (1980). Such additives are generally nontoxic to the recipients at the dosages and concentrations employed, hence, the excipients, diluents, carriers and so on are pharmaceutically acceptable.

The compositions can take the form of solutions, suspensions, emulsions, powders, sustained-release formulations, depots and the like. Examples of suitable carriers are described in “Remington's Pharmaceutical Sciences,” Martin. Such compositions will contain an effective amount of the biopolymer of interest, preferably in purified form, together with a suitable amount of carrier so as to provide the form for proper administration to the patient. As known in the art, the formulation will be constructed to suit the mode of administration.

Buffering agents help to maintain the pH in the range which approximates physiological conditions. Buffers are preferably present at a concentration ranging from about 2 mM to about 50 mM. Suitable buffering agents for use with the instant invention include both organic and inorganic acids, and salts thereof, such as citrate buffers (e.g., monosodium citrate-disodium citrate mixture, citric acid-trisodium citrate mixture, citric acid-monosodium citrate mixture etc.), succinate buffers (e.g., succinic acid monosodium succinate mixture, succinic acid-sodium hydroxide mixture, succinic acid-disodium succinate mixture etc.), tartrate buffers (e.g., tartaric acid-sodium tartrate mixture, tartaric acid-potassium tartrate mixture, tartaric acid-sodium hydroxide mixture etc.), fumarate buffers (e.g., fumaric acid-monosodium fumarate mixture, fumaric acid-disodium fumarate mixture, monosodium fumarate-disodium fumarate mixture etc.), gluconate buffers (e.g., gluconic acid-sodium glyconate mixture, gluconic acid-sodium hydroxide mixture, gluconic acid-potassium gluconate mixture etc.), oxalate buffers (e.g., oxalic acid-sodium oxalate mixture, oxalic acid-sodium hydroxide mixture, oxalic acid-potassium oxalate mixture etc.), lactate buffers (e.g., lactic acid-sodium lactate mixture, lactic acid-sodium hydroxide mixture, lactic acid-potassium lactate mixture etc.) and acetate buffers (e.g., acetic acid-sodium acetate mixture, acetic acid-sodium hydroxide mixture etc.). Phosphate buffers, carbonate buffers, histidine buffers, trimethylamine salts, such as Tris, HEPES and other such known buffers can be used.

Preservatives may be added to retard microbial growth, and may be added in amounts ranging from 0.2%-1% (w/v). Suitable preservatives for use with the present invention include phenol, benzyl alcohol, m-cresol, octadecyldimethylbenzyl ammonium chloride, benzyaconium halides (e.g., chloride, bromide and iodide), hexamethonium chloride, alkyl parabens, such as, methyl or propyl paraben, catechol, resorcinol, cyclohexanol and 3-pentanol.

Isotonicifiers are present to ensure physiological isotonicity of liquid compositions of the instant invention and include polhydric sugar alcohols, preferably trihydric or higher sugar alcohols, such as glycerin, erythritol, arabitol, xylitol, sorbitol and mannitol. Polyhydric alcohols can be present in an amount of between about 0.1% to about 25%, by weight, preferably 1% to 5% taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols; amino acids, such as arginine, lysine, glycine, glutamine, asparagine, histidine, alanine, ornithine, L-leucine, 2-phenylalanine, glutamic acid, threonine etc.; organic sugars or sugar alcohols, such as lactose, trehalose, stachyose, arabitol, erythritol, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thiosulfate; low molecular weight polypeptides (i.e., <10 residues); proteins, such as human serum albumin, bovine serum albumin, gelatin or immunoglobulins; hydrophilic polymers, such as polyvinylpyrrolidone, saccharides, monosaccharides, such as xylose, mannose, fructose or glucose; disaccharides, such as lactose, maltose and sucrose; trisaccharides, such as raffinose; polysaccharides, such as, dextran and so on. Stabilizers can be present in the range from 0.1 to 10,000 w/w per part of biopolymer.

Additional miscellaneous excipients include bulking agents, (e.g., starch), chelating agents (e.g., EDTA), antioxidants (e.g., ascorbic acid, methionine or vitamin E) and cosolvents.

Non-ionic surfactants or detergents (also known as “wetting agents”) may be added to help solubilize the therapeutic agent, as well as to protect the therapeutic protein against agitation-induced aggregation, which also permits the formulation to be exposed to shear surface stresses without causing denaturation of the protein. Suitable non-ionic surfactants include polysorbates (20, 80 etc.), polyoxamers (184, 188 etc.), Pluronic® polyols and polyoxyethylene sorbitan monoethers (TWEEN-20®, TWEEN-80® etc.). Non-ionic surfactants may be present in a range of about 0.05 mg/ml to about 1.0 mg/ml, preferably about 0.07 mg/ml to about 0.2 mg/ml.

The present invention provides liquid formulations of a biopolymer having a pH ranging from about 5.0 to about 7.0, or about 5.5 to about 6.5, or about 5.8 to about 6.2, or about 6.0, or about 6.0 to about 7.5, or about 6.5 to about 7.0.

The incubation of the amine-reacting proteoglycan with blood or tissue product can be carried out a specific pH in order to achieve desired properties. E.g., the incubation can be carried out at between a pH of 7.0 and 10.0 (e.g., 7.5, 8.0, 8.5, 9.0, and 9.5). Furthermore, the incubation can be carried out for varying lengths of time in order to achieve the desired properties.

### EXAMPLES

PK Analysis of His-iLZ-TRAIL and TRAIL_{PEG}

iLZ-TRAIL and TRAIL_{PEG} were prepared as described previously (Mol. Cancer Ther. 9(6):1719-1729 (2010)) and generously provided by Theraly Pharmaceuticals Inc. The PK of proteins were measured in cynomolgus monkeys. Male cynomolgus monkeys (4-5 kg, Korea Research Institute of Chemical Technology (KRICT), Daejeon, Korea) were fasted for 12 h before drug administration. iLZ-TRAIL and TRAILPEG (12.5 μg/kg, protein-based) were i.v. administered and blood samples (450 μL) were collected from the vein and mixed with 50 μL of sodium citrate (3.8% solution), followed by centrifugation at 2,500 g for 15 min at 4° C. The plasma samples were separated and stored at −70° C. Animal studies were carried out in accordance with the procedures outlined in the Guide for the Care and Use of Laboratory Animals and approved by KRICT. The concentration of TRAIL was determined by Human TRAIL/TNFSF10 Quantikine ELISA Kit (R&D Systems, Minneapolis, Minn.) and analyzed using GraphPad Prism 6 software (GraphPad Software, La Jolla, Calif.) based on a four-parameter logistic standard curve derived from iLZ-TRAIL and TRAL_{PEG}, respectively. PK parameters were obtained by non-compartmental analysis from WinNonlin (Pharsight Corporation, Mountain View, Calif.).

Cell Culture

The cell lines were purchased from American Type Culture Collection (Manassas, Va.). HT-29, SW620, HCT116, and MDA-MB-231 cells were maintained in RPMI 1640 medium (Sigma, St. Louis, Mo.) supplemented with 10% fetal bovine serum (FBS; Life Technology, Carlsbad, Calif.), 1% penicillin, and 1% streptomycin (Life Technology). Cells were cultured at 37° C. under an atmosphere of 5% CO₂. PC-3 and A549 cells were maintained in F-12K medium (Sigma) supplemented with 10% FBS, 1% penicillin, and 1% streptomycin. HEK293T cells were cultured in Modified Eagles Medium (MEM) (Sigma) supplemented with 10% FBS, 1% penicillin, and 1% streptomycin. Typically, 2×10⁵ cells per well were plated in 6-well plates for treatment of agents.

Cell Viability

A total of 1×10⁴ cells were plated in 0.1 mL in 96-well flat bottom plates and incubated for 24 h before being exposed to various stimuli. After incubation for the indicated times, 5 μg/mL MTT solution was added to each well and incubated for 1 h. After removal of the medium, 200 μL of DMSO was added to each well to dissolve the formazan crystals. The absorbance at 540 nm was determined using a microplate reader (Bio-Tek Instruments, Inc, Winooski, Vt.). Triplicate wells were assayed for each condition.

HCT116 Xenograft

All experiments involving tumor xenografts were performed according to protocols approved by the Johns Hopkins Animal Care and Use Committee and animal studies were undertaken in accordance with the rules and regulations. Freshly harvested HCT116 cells (3×10⁶ cells/mouse) were inoculated s.c. into BALB/c athymic mice (n=5). When tumor volume reached ˜50 mm³, mice were treated with TRAIL (8 mg/kg, i.v.) or TRAIL_{PEG} (8 mg/kg, i.v.) every 3 days for 2 weeks (total 4 times). Tumor volumes were monitored for 30 days after tumor cell administration. Tumor volumes were calculated using longitudinal (L) and transverse (W) diameters using V=(L*W²)/2, and tumor growth inhibition (TGI) percent values were calculated using the formula TGI %=(1−TVₛₐₘₚₗₑ/TV_{control})×100, where TV is tumor volume.

HT-29 Xenograft

The antitumor effects of TRAILPEG after HAC/DOX sensitizing were investigated in HT-29 tumor bearing mice (n=5). Briefly, freshly harvested HT-29 cells (5×10⁶ cells/mouse) were inoculated s.c. into BALB/c athymic mice. Treatment was initiated when the tumors reached a mean volume of 150 mm³. Mice were treated with three rounds of DOX or HAC/DOX (7 mg/kg, i.v.) combined with TRAILPEG (8 mg/kg, i.v.) for 10 days. The tumors were analyzed and calculated as described above. (n=5).

In Situ DNA Strand Break Labeling (TUNEL Assay)

Tumor tissues were recovered from euthanized animals. Sections (5 μm) were cut from 10% neutral buffered, formalin-fixed, paraffin-embedded tissue blocks. Apoptotic cell death in tumor tissues was visualized by performing TdT-mediated dUTP nick end labeling (TUNEL) assays according to the manufacturer instructions (Roche Mannheim, Germany).

Confocal Analysis

HT-29 cells grown on coverslips in 12-well plates were treated with indicated agents. The cells were fixed in 4% paraformaldehyde for 5 min and then washed with ice-cold PBS (pH 8.0) three times. Finally, the cells were mounted on slides for visualization under a Fluoview FV10i-DOC confocal microscope (Olympus Optical, Tokyo, Japan).

Antibodies and Western Blotting

Anti-caspase-8 (Cell Signaling Technology, Danvers, Mass., #9746), anti-cleaved PARP-1 (Cell Signaling Technology, #5625), anti-cleaved caspase-3 (Cell Signaling Technology, #9664), anti-cleaved caspase-9 (Cell Signaling Technology, #7237), anti-CD44 (Cell Signaling Technology, #5640), anti-p-JNK (Cell Signaling Technology, #4668), anti-p-p53 (Ser15 Cell Signaling Technology, #9284), anti-BCl-2 (Cell Signaling Technology, #2870), anti-p-BCL-2 (Cell Signaling Technology, #2875), anti-BCL-XL (Cell Signaling Technology, #2764), anti-DR4 (Abcam, Cambridge, Mass., #13890), anti-DR5 (Abcam, #47179), anti-c-Jun (Santa Cruz Biotechnology, Santa Cruz, Calif., sc-1694), or anti-β-actin (sc-47778) were used in the Western blot analysis. In generally, cells were lysed and sonicated briefly in ice-cold PBS buffer (1 mM PMSF, and 1 μg/ml each of aprotinin, leupeptin, and pepstatin A). Cell lysates were clarified by centrifugation, resolved by SDS-PAGE, and proteins on gels were transferred to nitrocellulose (Bio-Rad, Hercules, Calif.) using a semidry blotter (Bio-Rad). The membrane was blocked with 3% BSA in TBST (10 mM Tris-Cl, pH 8.0, 150 mM NaCl, 0.05% Tween-20) and incubated overnight at 4° C. with primary antibodies. Immunoblots were visualized by an enhanced chemiluminescence method.

siRNA Transfection

HT-29 cells were cultured in 6 well plates for 24 h and the cells were transfected with DR5 siRNA (Santa Cruz Biotechnology, Santa Cruz, Calif., sc-40237) or control siRNA for 48 h. Transfection was carried out using Lipofectamine 2000 reagent (Invitrogen) following to the manufacturer's instructions.

Quantitative RT (Reverse Transcription)-PCR

Total cellular RNA was purified from HT-29 cells using Trizol reagent (Life Technology) and subjected to amplification with SuperScript One-Step RT-PCR system (Life Technology). Real-time PCR was carried out using a StepOne™ Real-Time PCR System according to the manufacturer instructions (Life Technology). The mean cycle threshold value (Ct) from triplicate samples was used to calculate the gene expression. β-actin was used as an internal control to normalize the variability in expression. Experiment was repeated three times with identical results. The following specific primers sets that are consensus region among isoforms were used for PCR; DR4, forward 5′-TGT GAC TTT GGT TGT TCC GTT GC-3′ (SEQ ID NO: 1) and reverse 5′-ACC TGA GCC GAT GCA ACA ACA G-3′ (SEQ ID NO: 2); DR5, forward 5′-AAG ACC CTT GTG CTC GTT GT-3′ (SEQ ID NO: 3) and reverse 5′-AGG TGG ACA CAA TCC CTC TG-3′ (SEQ ID NO: 4); actin, forward 5′-TCC CTG GAG AAG AGC TAC GA-3′ (SEQ ID NO: 5) and reverse 5′-AGC ACT GTG TTG GCG TAC AG-3′ (SEQ ID NO: 6).

Flow Cytometry

Cells were harvested, washed with PBS, re-suspended in 75% ethanol in PBS, and kept at 4° C. for 30 min. Cells were re-suspended with 1 mM EDTA, 0.1% Triton-X-100 and 1 mg/ml RNAse A in PBS. The suspension was then analyzed on a FACSCaliber. To calculate percentage of cells in respective phases of the cell cycle, the DNA content frequency histograms were deconvoluted using the MultiCycle software (Phoenix Flow Systems, San Diego, Calif., USA).

DOX Distribution in HT-29 Xenograft Tumors

Mice bearing HT-29 xenograft tumors were intravenously administered with DOX (7 mg/kg) and HAC/DOX (containing 7 mg/kg equivalent doxorubicin) when tumors reached 300 mm³. At each selected time point, 3 mice in one group were euthanized by cervical dislocation. Whole blood was collected via cardiac puncture with a heparinized syringe. Tumors were dissected out and frozen at −70° C. immediately. Plasma samples were isolated from whole blood by centrifugation at 3000 g for 5 min. Tissues homogenates were prepared in 800 μL water using a Polytron homogenizer (Brinkman Instruments, Mississauga, Ontario, Canada), and then 200 μL of H₂SO₄ was added to the tissue homogenates. The solutions were then digested for 2 h at 60° C. After the vials cooled to room temperature, 100 μL of AgNO₃ was added. Then the samples were centrifuged at 12,000 g for 10 min, and the supernatant was counted in a fluorospectrometer (RF-5301, Shimadzu) at an excitation wavelength of 500 nm and emission wavelength of 558 nm. The concentration of doxorubicin in each tissue was calculated based on a calibration curve. The calibration curve was linear over the 0.02 and 2.00 μg/mL range with a correlation coefficient of R²=0.9993.

Death-Inducing Signaling Complex Immunoprecipitation.

After HT-29 cells achieved 80% confluence, the cells were pretreated with doxorubicin for 24 h and then incubated with 500 ng/mL Flag-TRAIL (Enzo Life Sciences, Farmington, N.Y.) for 30 min at 37° C. The cells were lysed with DISC IP lysis buffer (30 mM Tris, pH 7.4, 150 mM NaCl, 10% glycerol, 1% Triton X-100 with 1 mM PMSF, and 1 μg/mL each of aprotinin, leupeptin, and pepstatin A). Cell lysates were incubated with Flag (M2) beads (Sigma) overnight. The beads were subsequently washed three times with cold PBS, resolved onto SDS-PAGE gels and subjected to Western blot analysis.

Statistical Analysis

All data were analyzed by GraphPad Prism 6 (GraphPad Software, La Jolla, Calif.). Differences between two means were assessed by a paired or unpaired t-test. Differences among multiple means were assessed, as indicated, by one-way ANOVA, followed by Turkey's post-hoc test or by the Student's t-test as appropriate. Error bars represent S.D or S.E.M as indicated. P-values <0.05 were considered to be significant.

### Example 1

TRAIL_{PEG} improves pharmacokinetics and reduces tumor growth in TRAIL-sensitive tumor xenografts but does not influence apoptosis in TRAIL-resistant tumors.

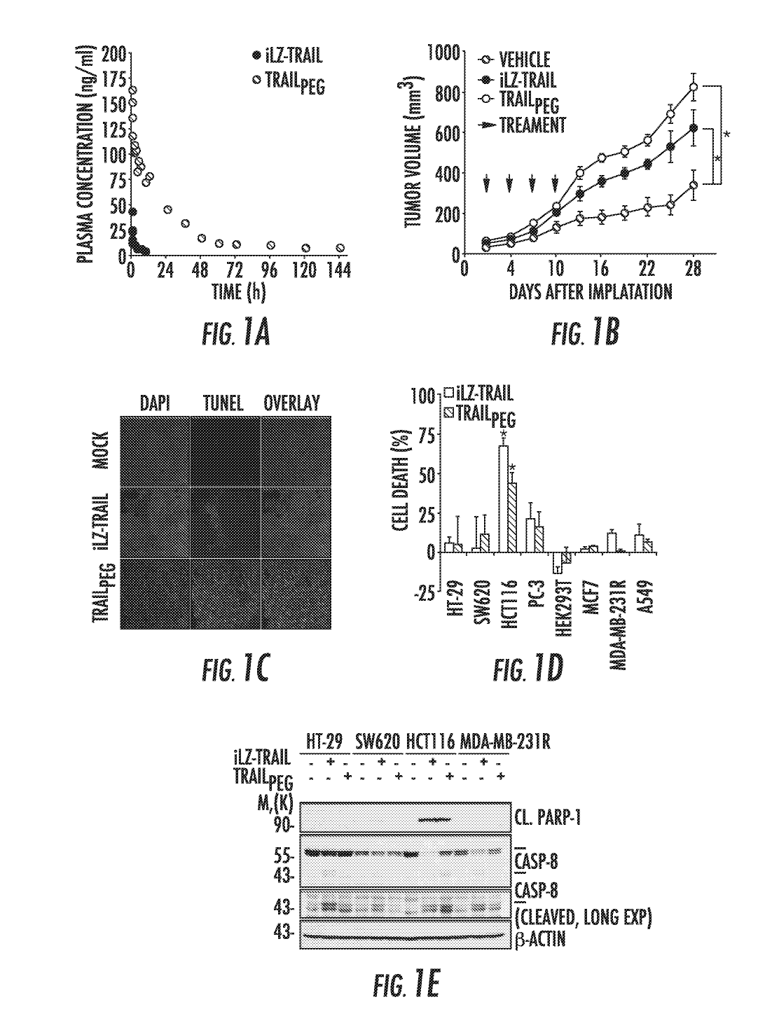

TRAIL_{PEG} engineered with a 20 kDa PEG molecule was synthesized as previously reported and used throughout the study. In addition to the earlier PK studies in rodents, we monitored the pharmacokinetics of TRAIL_{PEG} in cynomolgus monkeys (FIG. 1A). iLZ-TRAIL cleared from the blood with a t_{1/2} less than an hour. In contrast, TRAILPEG showed a 17-fold increase in t_{1/2} and a 38-fold increase in area under the curve (AUC) over iLZ-TRAIL and lasted in the blood up to 144 h after dosing (data not shown). To compare pharmacodynamics (PD) between iLZ-TRAIL and TRAILPEG, TRAIL variants were intravenously administered every 3 days for a total of 4 times in HCT116 xenografts when the tumor was palpable (50 mm3) (FIG. 1B). HCT116 is a human colon cancer cell line that is relatively sensitive to TRAIL-induced apoptosis. Compared to iLZ-TRAIL, TRAILPEG (200 μg, protein-based) showed increased tumor growth inhibition (TGI) values (at day 28 for iLZ-TRAIL and TRAILPEG; 27% and 58%, respectively). At the end of the study, tumor tissues were harvested and apoptotic cells in tumor sections were visualized by TdT-mediated dUTP nick and labeling (TUNEL) assay (FIG. 1C). TRAILPEG clearly showed tumor cell apoptosis in vivo compared to marginal signs in the iLZ-TRAIL-treated group.

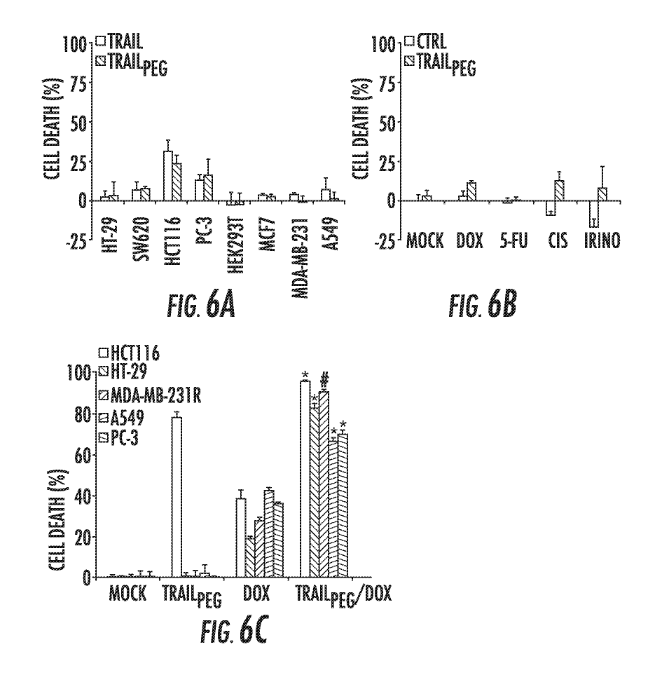

Next, we examined if the improved TRAIL stability of TRAILPEG contributes to apoptosis in TRAIL-resistant tumors. A panel of known TRAIL-resistant human tumor cell lines including colon (HT-29, SW620), prostate (PC3), breast (MDA-MB-231, MCF7) and lung (A549) as well as TRAIL-sensitive HCT116 and normal human kidney HEK293T cells were incubated with 1 μg/mL of iLZ-TRAIL or TRAILPEG for 3 h and 24 h in respective media. TRAIL sensitivities were expressed as induced cell death (%), calculated as the percentage relative to the untreated cells, and measured by MTT assays (FIG. 1D and FIG. 6). TRAILPEG provoked strong apoptosis only in TRAIL-sensitive HCT116 cells, like iLZ-TRAIL, as evidenced by upregulated cleavage of poly(ADP-ribose) polymerase 1 (PARP-1), a substrate of caspase-3 (FIG. 1E). This study validates that improved stability of TRAILPEG does not alter the DR-mediated apoptosis signaling in TRAIL-resistant tumors; thus an additional strategy to extend the t_{1/2} of TRAIL is needed to target both TRAIL-sensitive and -resistant tumors in vivo.

### Example 2

DOX/TRAIL_{PEG} potentiates DR-mediated apoptosis in TRAIL-resistant tumor cells.

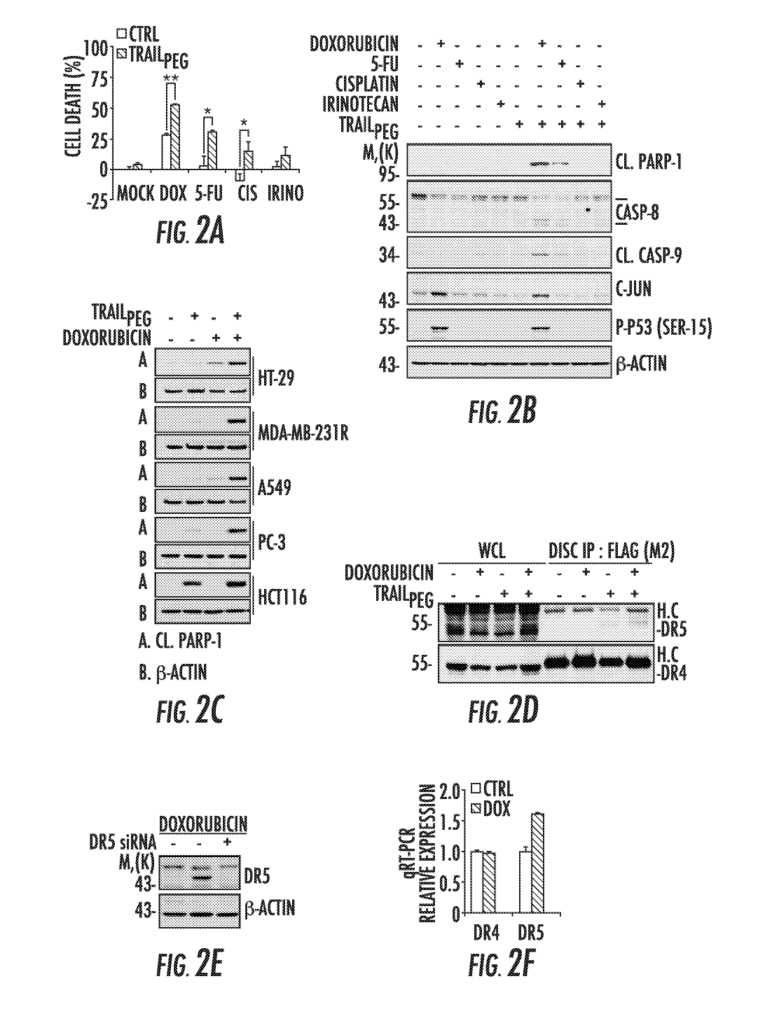

Accumulating reports suggest that various FDA-approved chemotherapies sensitize cancer cells to TRAIL-induced apoptosis. To identify synergism with TRAILPEG, common DNA damaging agents approved for colon cancer treatment, including doxorubicin (DOX), 5-fluorouracil (5-Fu), cisplatin (CIS), and irinotecan (IRINO), were incubated in TRAIL-resistant HT-29 cells as drug alone or with TRAILPEG and screened for apoptosis. Lower doses of agents (0.5 μg/mL of DOX, CIS; 1 μg/mL of 5-Fu and 0.6 μg/mL of IRINO) were pretreated in HT-29 cells for 24 h followed by an additional 24 h incubation with either drug alone or in combination with TRAIL_{PEG} (1 μg/mL). The low dose of drugs did not induce apoptosis as seen by the percentage of relative cell death (FIG. 6B). At high toxic doses (>10 μg/mL), most of the drug-treated cells were dead in 24 h (data not shown). When HT-29 cells were exposed to sub-lethal doses of DOX (2 μg/mL), 5-FU (10 μg/mL), CIS (2 μg/mL), or IRINO (3 μg/mL) combined with TRAIL_{PEG}, enhanced TRAIL-induced apoptosis was observed compared to drug alone (FIG. 2A). Among tested agents, DOX/TRAIL_{PEG} combination clearly enhanced apoptosis through the proteolytic activation of caspase-8 (Casp-8) and caspase-9 (Casp-9) and consequently cleaved PARP-1 in HT-29 cells (FIG. 2B). Treatment of DOX also led to the phosphorylation of p53 and the activation of c-jun, a downstream substrate of c-Jun N-terminal kinase (JNK).

Next, DOX/TRAIL_{PEG} combination treatment was examined for enhanced apoptosis in different TRAIL-resistant cells. Individually, TRAIL_{PEG} and DOX induced low levels of cleaved PARP-1 in TRAIL-resistant human tumor cell lines, including HT-29, MDA-MB-231 (breast), A549 (lung), and PC3 (prostate). When combined, cleaved PARP-1 expression was significantly increased in all TRAIL-resistant and TRAIL-sensitive cell lines examined, (FIG. 2C) and such synergism was correlated in cell death assays (FIG. 6C). To investigate if enhanced apoptosis by DOX/TRAIL_{PEG} is DR-mediated through death-inducing signaling complex (DISC) formation, TRAIL DISC immunoprecipitation (IP) was assessed in HT-29 cells after treatment of DOX, TRAIL or DOX/TRAIL followed by DR4 and DR5 Western blotting (FIG. 2D). Interestingly, TRAIL DISC demonstrated the recruitment of DR5, but not DR4, on the cellular membrane after DOX/TRAIL treatment. For further validation, HT-29 cells that were transfected with DR5 siRNA and treated with DOX had attenuated expression of DR5 (FIG. 2E). As examined by quantitative real-time PCR (qPCR), DOX increased DR5 mRNA by 60% in HT-29 cells compared to untreated cells, whereas DR4 mRNA levels did not change (FIG. 2F).

### Example 3

DOX/TRAIL_{PEG} accelerates proteolytic activation of caspases through DR5 upregulation in HT-29 cells.

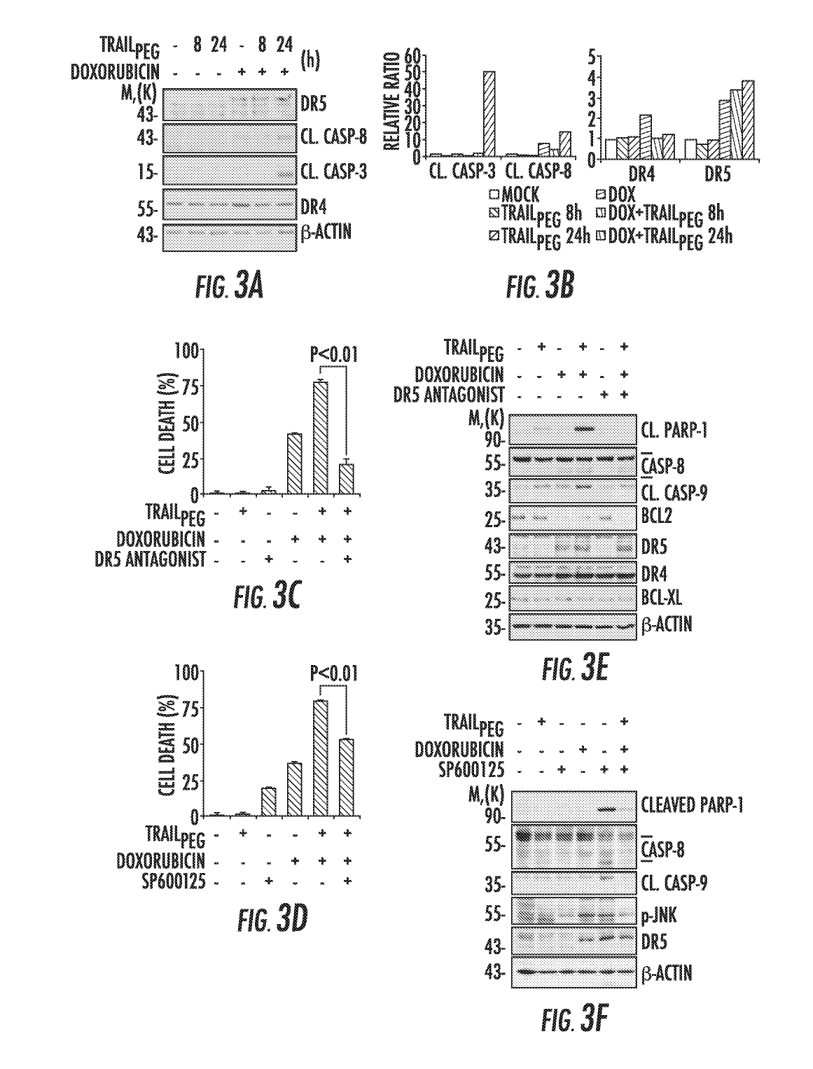

It has been reported that HT-29 cells are TRAIL-resistant because of low DR5 expression on the cellular membrane. In other reports, DOX has been demonstrated to sensitize TRAIL-induced apoptosis by affecting the cell surface localization of DR5 in colon cancer cells. To explore how DOX and DOX/TRAIL_{PEG} enhance apoptosis, HT-29 cells were treated with DOX or DOX/TRAIL_{PEG} at different time points. Pretreatment of DOX (2 μg/mL) activated Casp-8 when treated alone and Casp-3 when TRAIL_{PEG} was co-treated for 24 h (FIGS. 3A and 3B). Regardless of TRAIL_{PEG}, DOX upregulated DR5 expression (3 to 4-fold), but not DR4, at the protein level. TRAIL intrinsically binds to both DR4 and DR5, but we have shown that only altered levels of DR5 in HT-29 cells play a critical role in TRAIL-induced apoptosis while DR4 levels remained unchanged.

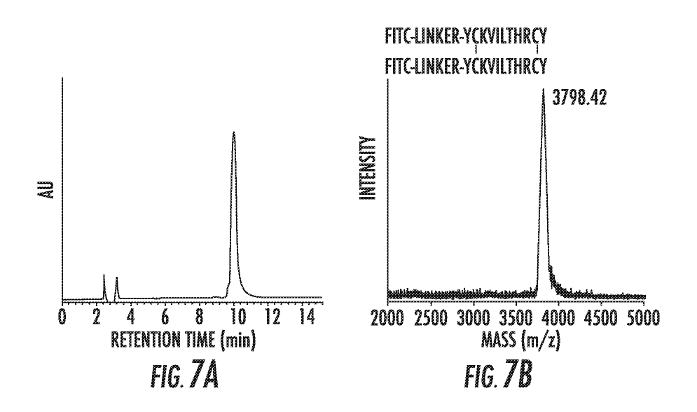

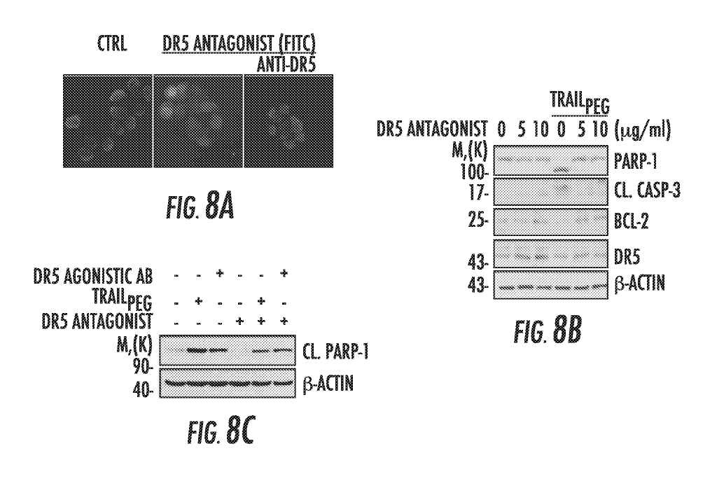

To assess if the enhanced DOX/TRAIL_{PEG}-induced apoptosis is due to altered DR5 expression, we synthesized a peptide-based dimeric DR5 antagonist (DR5-A) based on a reported sequence of YCKVILTHRCY (SEQ ID NO: 7) (FIG. 7 and FIG. 8A). The neutralizing efficacy of DR5-A was confirmed by treating HCT116 cells with DR5-A (5, 10 μg/mL) and TRAIL_{PEG} or DR5 agonistic antibody (FIG. 8B and FIG. 8C). Upon incubation, the DR5-A effectively blocked TRAIL_{PEG}-induced apoptosis by neutralizing DR5 as evidenced by the reduced expression of cleaved Casp-3 and PARP-1. With this antagonistic peptide, we investigated the extent of DR5 expression induced by DOX treatment and its effect on DOX/TRAIL_{PEG}-induced apoptosis in HT-29 cells. When TRAILPEG was co-treated with both DOX and DR5-A to HT-29 cells, cell death evoked by the DOX/TRAIL_{PEG} treatment was significantly inhibited by 70% compared to that of cells without a DR5-A treatment (FIG. 3C). Blocking DR5 substantially decreased the proteolytic activation of Casp-8, Capse-9 and PARP-1 cleavage in cells treated with DOX/TRAIL_{PEG} while showing no effect on BCL2/BCL-XL expression that was mainly reduced by DOX (FIG. 3E).

It has been reported that JNK mediates DOX- or TRAIL-induced apoptosis in cancer cells. In addition, activation of the JNK pathway leads to DR upregulation in multiple tumor cells including colon cancer. We hypothesized that the DOX-induced DR5 upregulation in HT-29 cells is p53-independent and JNK pathway dependent. To study this, HT-29 cells were treated with DOX and TRAIL_{PEG} alone or in combination with SP600125 (20 μM), a JNK inhibitor. Consequently, inhibition of JNK phosphorylation reduced DOX and TRAIL_{PEG}-induced cell death by 35% (FIG. 3D) and suppressed proteolytic activation of Casp-8, Casp-9 and PARP-1 cleavages (FIG. 3F). However, SP600125 had no effect on regulating DR5, indicating DOX-induced DR5 upregulation is not stimulated by the JNK pathway. This suggests that JNK partially mediates DOX/TRAIL_{PEG}-induced apoptosis but is not involved in DR5 upregulation in HT-29 cells.

### Example 4

Tumor-homing HAC/DOX but not free DOX accumulates DOX concentrations in tumor tissues in vivo.

In many cases, select anticancer agents acting as TRAIL sensitizers in vitro were not fully validated in animal models and when in vivo efficacy was demonstrated, relatively high doses of drugs were needed to effectively sensitize TRAIL-resistant tumors in vivo. However, such exceedingly high doses of chemotherapeutic agents as TRAIL sensitizers are not clinically practical. One effective way to utilize such toxic agents as a sensitizer while minimizing systemic toxicity in vivo is using a tumor-homing drug delivery system. We previously optimized a hyaluronic acid-based conjugate (HAC), a tumor-homing nanocarrier system comprised of biocompatible hyaluronic acid, that can deliver small molecules to the intracellular space of cancer cells via CD44 receptors with reduced systemic toxicity (Biomaterials 33(26):6186-6193 (2012); Biomaterials 31(1):106-114 (2010)). Importantly, this targeted, intracellular delivery was observed and verified in different in vivo cancer models, ranging from colon and melanoma to head and neck (ACS Nano 5(11):8591-8599 (2011); Colloids Surf B Biointerfaces 99:82-94 (2012); J Control Release 172(3):653-661 (2103)).

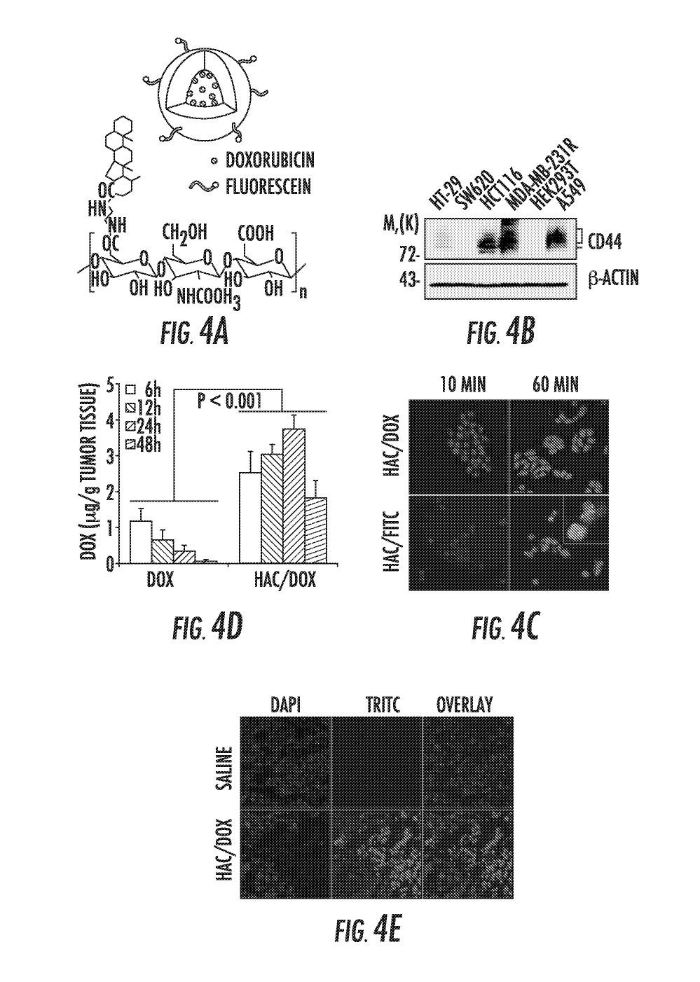

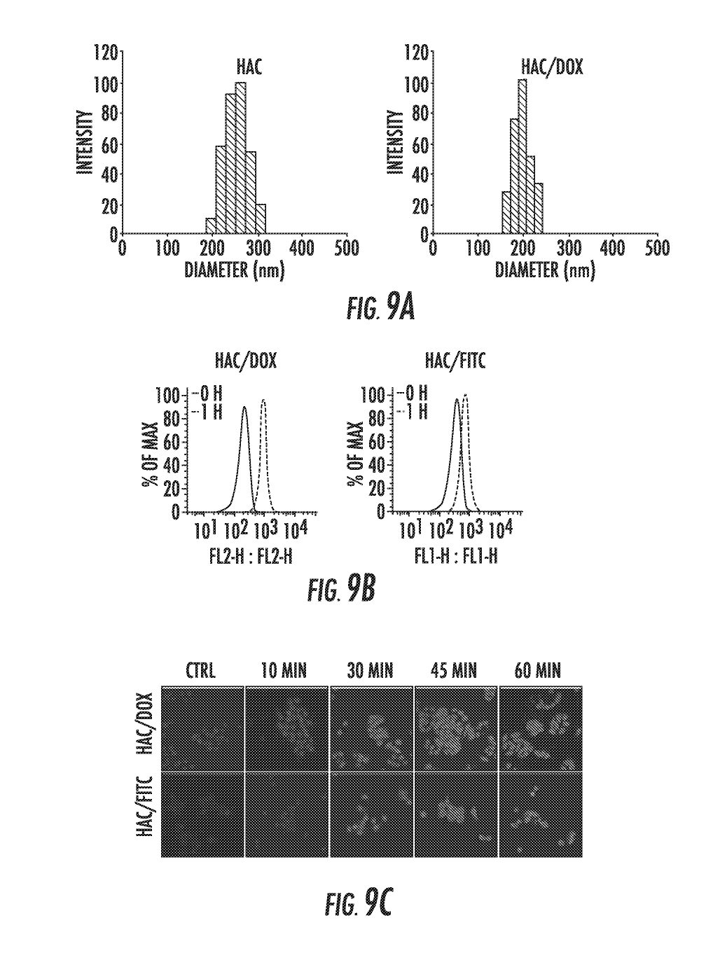

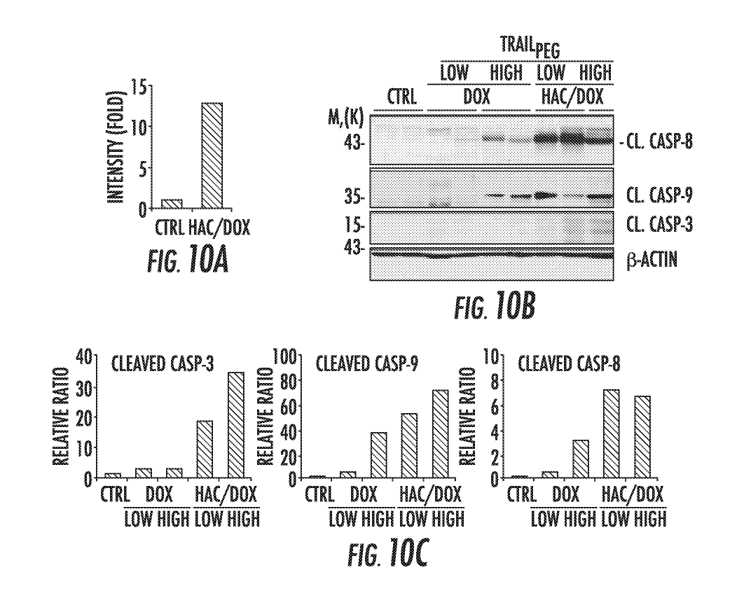

In aqueous solutions, the HAC structure can self-assemble into a nanocarrier sequestering the hydrophobic/amphiphilic molecules to the center of the particle. Because of HAC's abundant functional groups, the surface of HAC can be modified with fluorophores or other detectable moieties for tracking and imaging in cells and in vivo (Nano Lett 12(7):3613-3620 (2012)). The schematic diagram and chemical structure of HAC is described in FIG. 4A. CD44 expression and therefore HAC drug delivery is dependent on cell types. Among the tested cells, HT-29, HCT116, MDA-MB-213 and A549 tumor cells express CD44 whereas SW620 and HEK293T cells do not express high levels of CD44 (FIG. 4B). DOX is well-encapsulated in HAC (HAC/DOX) with high loading contents (21%, wt) and loading efficiency (71%) with mean diameter of 206 nm in PBS (10 mM, pH 7.4) (FIG. 9A). When DOX was incorporated in HAC labeled with fluorescein molecules and treated to HT-29 cells, HAC/DOX showed rapid cellular uptake after 10 min of incubation and saturated at 1 h (FIG. 4C and FIG. 9B). Importantly, HAC/DOX promptly burst releases the incorporated DOX inside the cell, as evidenced by the restored quenched fluorescence of DOX in microscopy and FACS data (FIG. 9C). HAC was shown to be non-toxic in our previous studies. The tumor-homing delivery of DOX by HAC was studied in tumor xenograft models. DOX concentration in plasma and tumor tissues was quantified by fluorescence absorbance followed by an extraction process. When HAC/DOX and the same dose of DOX dissolved in saline was intravenously injected in HT-29 xenografts bearing approximately 300 mm³ tumors, HAC/DOX delivered more DOX in the harvested tumor tissues than free DOX. The concentration of DOX in the tumor region gradually decreased with time at 6 h post drug administration (FIG. 4D). In contrast, HAC/DOX markedly increased DOX accumulation in the tumor region from 6 h to 24 h and maintained accumulation 48 h post-injection. HAC/DOX demonstrated 12-fold and 55-fold increased accumulation of DOX in isolated tumors at 24 h and 48 h, respectively, compared to that of DOX alone. To confirm DOX distribution in tumors, harvested tumor sections isolated at 48 h were stained with DAPI and visualized by confocal microscopy (FIG. 4E and FIG. 10A). As expected, HAC/DOX treated tissues showed an obvious sign of DOX accumulation compared to the DOX treatment alone.

### Example 5

A tumor-homing HAC/DOX combined with long-acting TRAIL_{PEG} potentiates apoptosis in TRAIL-resistant tumor xenografts with reduced systemic toxicity.

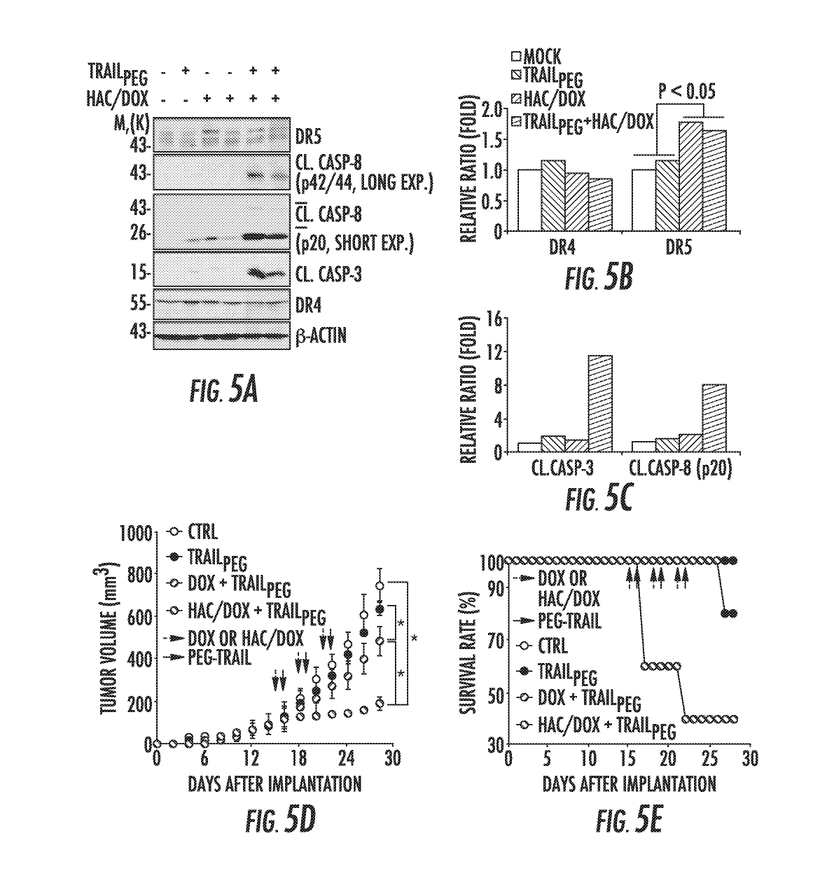

After confirming that HAC/DOX predominantly accumulates in tumors in vivo, the next study examined if HAC/DOX and TRAIL_{PEG} combination effectively upregulates DR5 and initiates apoptosis in vivo as demonstrated in vitro. When tumor volumes reached 200 mm³, HT-29 xenografts were intravenously treated with TRAIL_{PEG}, HAC/DOX and HAC/DOX/TRAILpEG (n=3). Because a caspase cascade was potentiated in HT-29 cells only when DOX was pretreated in the TRAIL_{PEG} treatment (FIGS. 2B and 2C), mice were treated by HAC/DOX (5 mg/kg, DOX-based) 24 h before TRAIL_{PEG} treatment. After 24 h of TRAIL_{PEG} treatment, the expression of DR5 and DR4 as well as Casp-8 and Casp-3 were analyzed in harvested tumor tissues. In accordance with cellular studies, HAC/DOX treatment increased the protein expression of DR5 in tumors by 70% in vivo while DR4 levels remained unchanged (FIGS. 5A and 5B). In particular, neither HAC/DOX nor TRAIL_{PEG} alone failed to initiate a caspase cascade. Casp-8 and Casp-3 were strongly activated only when the HAC/DOX and TRAIL_{PEG} were co-treated (FIG. 5C). To find a dose range in mice models, two TRAIL_{PEG} formulations with different DOX concentrations, low (2 mg/kg, DOX-based, Dox_{low}) and high (7 mg/kg, close to maximum tolerated dose, Dox_{high}), were injected in HT-29 xenografts when tumor volumes reached 200 mm3 followed by TRAIL_{PEG} treatment. Each tumor was harvested and analyzed by immunoblotting after 24 h of TRAIL_{PEG} treatment (FIG. 10B). After a single treatment, DOX at the low dose marginally increased the expression of cleaved Casp-9 and Casp-8 but showed some enhanced expression at the high DOX dose. Interestingly, neither low nor high DOX doses alone altered Casp-3 levels, an indicator of apoptosis. In contrast, HAC/DOX combined with TRAIL_{PEG} clearly initiated the caspase cascade by regulating cleaved Casp-9 and Casp-8 at both low and high DOX doses. HAC/DOX with low and high DOX concentrations significantly enhanced Casp-3 activation compared to DOX alone (for DOXl_{ow}, DOX_{high}, HAC/DOX_{low}, HAC/DOX_{high} vs. control, 2, 2, 13, and 24-fold) (FIG. 10C). In contrast to in vitro results, free DOX at the high dose was shown to marginally alter expression of initiator caspase and not executional caspase, Casp-3, when combined with TRAIL_{PEG} in vivo.

After confirming the necessary condition to generate TRAIL-induced apoptosis in TRAIL_{PEG}-resistant tumors in vivo, the drug efficacy and safety of the HAC/DOX and TRAIL_{PEG} combination in HT-29 xenografts was examined. rhTRAIL was excluded from the study, because rhTRAIL is less potent then TRAIL_{PEG}. DOX and HAC/DOX alone were also ruled out for the in vivo studies, because they do not efficiently induce apoptosis in HT-29 tumor models as presented earlier. When tumor volumes reached 150 mm³, mice were intravenously treated with TRAIL_{PEG} alone or with DOX and HAC/DOX at a 7 mg/kg DOX dose every 3 days for a total of 3 times as indicated in FIG. 5D. As demonstrated by in vitro studies, TRAIL_{PEG} alone marginally altered tumor growth. In contrast, the two TRAIL_{PEG} combinations suppressed tumor growth. TGI values were significantly decreased by the HAC/DOX and TRAIL_{PEG} combination throughout the study period (at day 28, for TRAIL_{PEG}, DOX and TRAIL_{PEG}, HAC/DOX and TRAIL_{PEG}; 14, 34, and 75% respectively). It should be noted that 60% of mice treated with a high dose of free DOX died during the treatment cycle due to the toxicity of the agent (FIG. 5E). Although carrying the same amount of high dose, HAC/DOX demonstrated a significantly improved tolerability in terms of survival rate, allowing the use of a necessary high dose of TRAIL sensitizer in vivo.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising pegylated TRAIL peptide (TRAIL_{PEG}) or functional fragments or variants thereof.

2. The composition of claim 1, wherein the TRAIL_{PEG} comprises amino acids 39-281 of the human TRAIL polypeptide.

3. The composition of claim 1, wherein the TRAIL_{PEG} comprises amino acids 41-281, 91-281, 92-281, 95-281, and 114-281, of the human TRAIL polypeptide.

4. The composition of claim 1, wherein the TRAIL_{PEG} functional fragment comprises amino acids 132-281, amino acids 95-281, or amino acids 114-281 to include C-terminal of the human TRAIL polypeptide that includes the receptor binding domain.

5. A pharmaceutical composition comprising an effective amount the TRAIL_{PEG} composition of claim 1 and pharmaceutically acceptable nanoparticle carrier.

6. A pharmaceutical composition comprising an effective amount the TRAIL_{PEG} composition of claim 1 and an effective amount of one or more TRAIL sensitizing compounds and pharmaceutically acceptable nanoparticle carrier.

7. The pharmaceutical composition of claim 6, wherein the sensitizing compounds are selected from the group consisting of doxorubicin, 5-fluorouracil, irinotecan or cisplatin.

8. The pharmaceutical composition of claim 6, wherein the pharmaceutically acceptable nanoparticle carrier comprises a hyaluronic acid-based conjugate (HAC).

9. The pharmaceutical composition of claim 7, wherein the TRAIL sensitizer comprises an HAC nanoparticle and doxorubicin.

10. A method for the treatment of cancer in a subject in need thereof, comprising administering to the subject, an effective amount of the composition of claim 6 to induce apotosis in the cancer of the subject.

11. The method of claim 10, wherein the nanoparticle comprising an effective amount of one or more TRAIL sensitizing compounds comprises doxorubicin, 5-fluorouracil, irinotecan or cisplatin.

12. The method of claim 10, wherein the nanoparticle comprises a hyaluronic acid polymer comprising a CD44 binding peptide.


SEQUENCE LISTING (6 sequences)

SEQ ID NO: 1 - DNA, 23 bp, Artificial Sequence
  synthetic sequence
tgtgactttggttgttccgttgc

SEQ ID NO: 2 - DNA, 22 bp, Artificial Sequence
  synthetic sequence
acctgagccgatgcaacaacag

SEQ ID NO: 3 - DNA, 20 bp, Artificial Sequence
  synthetic sequence
aagacccttgtgctcgttgt

SEQ ID NO: 4 - DNA, 20 bp, Artificial Sequence
  synthetic sequence
aggtggacacaatccctctg

SEQ ID NO: 5 - DNA, 20 bp, Artificial Sequence
  synthetic sequence
tccctggagaagagctacga

SEQ ID NO: 6 - DNA, 20 bp, Artificial Sequence
  synthetic sequence
agcactgtgttggcgtacag